(19) United States
(12) Patent Application Publication
(10) Pub. No.: US 2022/0213151 A1
(43) Pub. Date: Jul. 7, 2022

(54) COMPOSITIONS AND METHODS FOR TREATMENT OF DISTENTABLE TISSUES

(71) Applicant: Aurora Oncology, Aurora, CO (US)

(72) Inventor: Shawn ZINNEN, Aurora, CO (US)

(21) Appl. No.: 17/602,193

(22) PCT Filed: Apr. 22, 2020
(86) PCT No.: PCT/US2020/029396

§ 371 Date: Oct. 7, 2021

Related U.S. Application Data
(60) Provisional application No. 62/837,533, filed on Apr. 23, 2019.

(51) Int. Cl. C07K 14/34; C07K 14/485; A61P 35/00

(52) U.S. Cl. CPC C07K 14/34; C07K 14/485; A61K 38/00; C07K 2319/55; A61P 35/00

(57) ABSTRACT

Provided herein are compositions and methods for treating cancer in a distentable tissue, for example, a bladder, a uterus, peritoneum, omentum, or eye. Useful compositions include DT-EGF fusion proteins as described herein.

### RELATED APPLICATIONS

This application claims the benefit of U.S. Provisional application No. 62/837,533 titled “Compositions and Methods for Treatment of Distentable Tissues” filed Apr. 23, 2019 which is incorporated herein by reference.

### SEQUENCE LISTING

An official copy of the sequence listing is submitted concurrently with the specification electronically via EFS-Web as an ASCII formatted sequence listing with a file name of “10037.001WO1_ST25”, a creation date of Apr. 22, 2020, and a size of about 18.8 kilobytes. The sequence listing contained in this ASCII formatted document is part of the specification and is herein incorporated by reference in its entirety.

### TECHNICAL FIELD

The present disclosure relates to new therapeutics and methods for treatment of distentable tissues, particularly, treatment of cancer in distenable tissues.

### BACKGROUND

Drugs have specificity for disease cells or are designed to specifically target elements of the diseased cells. All drugs, regardless of mechanism, must bind and/or be taken up by their respective target cell of interest. Unfortunately, in the practical settings of disease treatment, drugs rarely if ever bind all targets or achieve uniform targeting of the whole population of diseased cells. This is one cause of incomplete response and recurrence of disease.

### BRIEF SUMMARY

Provided herein are pharmaceutical compositions and methods for treating cancer in distenable tissues.

In some embodiments, a pharmaceutical composition comprising a diphtheria toxin-epidermal growth factor (DT-EGF) fusion protein of structure A-X-Y-Z, wherein: A is 0-5 amino acid residue N-terminal addition to the front of the diphtheria toxin sequence; X is a diphtheria toxin fragment or mutated fragment that maintains catalytic activity; Y is sequence of amino acids from 0-20 aa in length that connects the carboxy terminus of X to the amino terminus of Z; and Z is an epidermal growth factor sequence or mutant of that sequence that maintains or improves binding affinity for epidermal growth factor receptor.

In some aspects, the composition has a pH of at least about 7.0, or a pH of at least about 7.4, or a pH of at least about 8.0.

In some embodiments, the composition is formulated with: 10 mM PO₄ and 150 mM NaCl at pH 8.0. In some aspects, the composition is formulated with 100 mM glucose.

In some aspects, A is a single alanine.

In some aspects, X is (glycosylation sites removed)
gaddvvdss ksfvmenfas yhgtkpgyvd siqkgiqkpk sgtqgnyddd wkgfystdnk ydaagysvdn enplsgkagg vvkvtypglt kvlalkvdna etikkelgls lteplmeqvg teefikrfgd gasrvvlslp faegsssvey innweqakal sveleinfet rgkrgqdamy eymaqacagn rvrrsvgssl scinldwdvi rdktktkies lkehgpiknk msespaktvs eekakqylee fhqtalehpe lselktvtgt npvfaganya awavnvaqvi dsetadnlek ttaalsilpg igsvmgiadg avhhnteeiv aqsialsslm vaqaiplvge lvdigfaayn fvesiinlfq vvhnsynrpa yspghktqpf (SEQ ID NO: 1)

In some aspects, X is (wild type sequence)
gaddvvdss ksfvmenfss yhgtkpgyvd siqkgiqkpk sgtqgnyddd wkgfystdnk ydaagysvdn enplsgkagg vvkvtypglt kvlalkvdna etikkelgls lteplmeqvg teefikrfgd gasrvvlslp faegsssvey innweqakal sveleinfet rgkrgqdamy eymaqacagn rvrrsvgssl scinldwdvi rdktktkies lkehgpiknk msespnktvs eekakqylee fhqtalehpe lselktvtgt npvfaganya awavnvaqvi dsetadnlek ttaalsilpg igsvmgiadg avhhnteeiv aqsialsslm vaqaiplvge lvdigfaayn fvesiinlfq vvhnsynrpa yspghktqpf (SEQ ID NO: 2)

In some aspects, X is a modified sequence that maintains catalytic activity while also evading immune response.

In some aspects, Y is ha, pw, lp, aa, or gg.

In some aspects, Y is:
a. A short sequence of 0-20 amino acids designed to be targeted by lysosomal or intracellular proteases
b. A short sequence of 0-20 amino acids designed to minimize negative or inhibitory interactions of X and Z sequences.

In some aspects, Z is wild type human EGF sequence.

In some aspects, Z is a mutant that increases affinity for the EGF receptor. In some aspects, Z is:
wnsYsecp PsYdgyclhd gvcRyieald Syacncvvgy Agercqyrdl RwwGRr (SEQ ID NO: 3).

In some aspects, XYZ is:
gaddvvdss ksfvmenfas yhgtkpgyvd siqkgiqkpk sgtqgnyddd wkgfystdnk ydaagysvdn enplsgkagg vvkvtypglt kvlalkvdna etikkelgls lteplmeqvg teefikrfgd gasrvvlslp faegsssvey innweqakal sveleinfet rgkrgqdamy eymaqacagn rvrrsvgssl scinldwdvi rdktktkies lkehgpiknk msespaktvs eekakqylee fhqtalehpe lselktvtgt npvfaganya awavnvaqvi dsetadnlek ttaalsilpg igsvmgiadg avhhnteeiv aqsialsslm vaqaiplvge lvdigfaayn fvesiinlfq vvhnsynrpa yspghktqpf lpwnsYsecp PsYdgyclhd gvcRyieald Syacncvvgy Agercqyrdl RwwGRr (SEQ ID NO: 4)

In some aspects, AXYZ is:
agaddvvdss ksfvmenfas yhgtkpgyvd siqkgiqkpk sgtqgnyddd wkgfystdnk ydaagysvdn enplsgkagg vvkvtypglt kvlalkvdna etikkelgls lteplmeqvg teefikrfgd gasrvvlslp faegsssvey innweqakal sveleinfet rgkrgqdamy eymaqacagn rvrrsvgssl scinldwdvi rdktktkies lkehgpiknk msespaktvs eekakqylee fhqtalehpe lselktvtgt npvfaganya awavnvaqvi dsetadnlek ttaalsilpg igsvmgiadg avhhnteeiv aqsialsslm vaqaiplvge lvdigfaayn fvesiinlfq vvhnsynrpa yspghktqpf lpwnsdsecp lshdgyclhd gvcmyieald kyacncvvgy igercqyrdl kwwelr (SEQ ID NO: 5) (A-dmDT390-EGF)

In some aspects, AXYZ is:
agaddvvdss ksfvmenfas yhgtkpgyvd siqkgiqkpk sgtqgnyddd wkgfystdnk ydaagysvdn enplsgkagg vvkvtypglt kvlalkvdna etikkelgls lteplmeqvg teefikrfgd gasrvvlslp faegsssvey innweqakal sveleinfet rgkrgqdamy eymaqacagn rvrrsvgssl scinldwdvi rdktktkies lkehgpiknk msespa ktvs eekakqylee fhqtalehpe lselktvtgt npvfaganya awavnvaqvi dsetadnlek ttaalsilpg igsvmgiadg avhhnteeiv aqsialsslm vaqaiplvge lvdigfaayn fvesiinlfq vvhnsynrpa yspghktqpf lpwnsYsecp PsYdgyclhd gvcRyieald Syacncvvgy Agercqyrdl RwwGRr (SEQ ID NO: 6)

The above compositions can be used in the methods described below and throughout the description. In some aspects, provided herein is a pharmaceutical composition for use in the manufacture of a medicament for use in the methods disclosed herein. In some aspects, the pharmaceutical composition, i.e. the targeted drug treatment, comprises SEQ ID NO: 5 or SEQ ID NO: 6. In some aspects, the targeted drug treatment is SEQ ID NO: 5. In some aspects, the targeted drug treatment is SEQ ID NO: 6.

Provided herein is a method to treat cancer in a distentable organ with an instilled volume that maximizes exposure of cellular surface targets to a targeted drug treatment. In some aspects, the targeted drug treatment is SEQ ID NO: 5. In some aspects, the targeted drug treatment is SEQ ID NO: 6.

Provided herein is a method to treat cancer in a distentable organ with an instilled volume comprised of a formulation and set of conditions that maximizes exposure of cellular surface targets to a targeted drug treatment. In some aspects, the targeted drug treatment is SEQ ID NO: 5. In some aspects, the targeted drug treatment is SEQ ID NO: 6.

Provided herein is a method to treat cancer in a distentable organ with an instilled volume comprised of a formulation and set of conditions in a pre-dosing treatment that maximizes exposure of cellular surface targets to a targeted drug treatment. In some aspects, the targeted drug treatment is SEQ ID NO: 5. In some aspects, the targeted drug treatment is SEQ ID NO: 6.

Provided herein is a method to treat cancer in a distentable organ with an instilled volume comprised of a formulation and set of conditions in a pre-dosing treatment that removes constituents that inhibit binding and exposure of cellular surface targets to a targeted drug treatment. In some aspects, the targeted drug treatment is SEQ ID NO: 5. In some aspects, the targeted drug treatment is SEQ ID NO: 6.

Provided herein is a method to treat cancer in a distentable organ with an instilled volume comprised of a formulation and set of conditions in a pre-dosing treatment that maximizes treatment efficacy by mechanisms other than improved binding of drug to target. An example of a pre-dosing treatment would be as that shown in FIG. 1—e.g. instill up to 500 mL of sterile isotonic saline with 10 mM Citrate pH 6.0, 37° C., 0.01% sodium dodecylsulfate and hold for 15 minutes, subsequently empty bladder and flush and empty up to 3× with 500 mL sterile isotonic saline. This causes a breakdown and removal of glycocalyx and other binding inhibitors.

Also provided herein is a method to treat cancer in a distentable organ with an instilled volume comprised of a formulation and set of conditions in a post-dosing treatment that maximizes treatment efficacy by mechanisms other than improved binding of drug to target. An example of a post-dosing treatment includes instilling up to 500 mL and holding for up to 1 hour a solution of 0.25% Acetic Acid at pH 4.5 and 40° C., subsequently emptying the bladder and flushing and emptying 3× with up to 500 mL sterile isotonic saline. This enables drug release from endosome facilitated by acid and the heat induced increase in chaperone proteins increased cytoplasmic drug concentration and activity.

The cancer can be in the bladder, the pleura, the uterus, the peritoneum, the eye, or the omentum. In some aspects, the cancer is in the bladder.

In some aspects, the pharmaceutical composition is a protein-toxin fusion of Diphtheria toxin to Epidermal Growth factor, a protein-toxin fusion of Pseudomonas Exotoxin A to EpCAM, or the protein-toxin fusion of IL-2 with diphtheria toxin known as Denileukin diftitox or ONTAK.

### DESCRIPTION OF THE FIGURES

FIG. 1 provides a schematic demonstrating the treatment of cancer such as bladder cancer by distension.

FIG. 2 demonstrates binding measurements and analyses of two DTEGF fusion-toxins to EGF by Surface Plasmon Resonance (SPR). SPR sensorgram (black lines) and the results from non-linear least squares regression analysis of the data (red lines) are shown. The graphs are the sensorgrams and the global fit to a 1:1 binding model for duplicate injections of six analyte concentrations ranging between 300 nM to 1.23 nM against captured rhu EGFR (ligand).

FIG. 3 shows A-dmDT390-EGF and A-dmDT390-EGF-Kd+/rhuEGFR interactions, including kₐ, k_{d}, Rₘₐₓ, and K_{D}, as buffer conditions are adjusted.

FIG. 4 demonstrates cytotoxicity of A-dmDT390-EGF (B05) and A-dmDT390-EGF-Kd+(BO1) in HTB9Luc cells after 15 minutes of treatment.

FIG. 5 depicts improved HTB9 cytotoxicity of A-dmDT390-EGF achieved by including 10 mM or 100 mM glucose in the treatment buffer.

### DETAILED DESCRIPTION

Provided herein are novel compositions of fusion proteins composed of natural occurring and modified amino acid sequences of Diphtheria Toxin (DT) and Epidermal Growth Factor (EGF) and the amino acid sequence that joins them. DT-EGF fusion proteins have been developed as potential therapeutics to treat cancer and disease driven by cells that overexpress Epidermal Growth Factor Receptor (EGFR). The methods and compositions disclosed herein can be applied to any DT-EGF fusion protein to minimize or eliminate immune inactivation and to improve its efficacy by enhancing the activities of the respective drug moieties: DT's catalytic activity driving ribosomal inactivation and facilitating endosomal release, EGF's binding of receptors and cellular uptake, and linking amino acid sequences ability to maximize the independence of the DT and EGF activities. The linker may include an amino acid sequence that provides susceptibility to intracellular protease to enable release of the DT from the EGF moiety once inside the target cell. The methods and compositions disclosed herein provide a novel means of avoiding immune inactivation of DT-EGF, enhancing specific binding to target receptors on diseased cells, enhancing targeted uptake and intracellular release, enhancing intracellular toxicity of the DT moiety, and minimizing interference of the DT with the EGF moiety functions (and vice versa).

To further extend the efficacy of fusion protein drugs like DT-EGF, and any drug that does not depend upon passive diffusion for activity, is provided herein a novel method for treatment of diseases of distentable tissues, where instillation or injection into a distentable tissue such as bladder, pleura, uterus, peritoneum, omentum, or eye is the route of administration; a distentable tissue is any tissue that can hold a defined volume of fluid for a minimum of 5 minutes and may or may not contain transitional epithelium. The method can be applied to any therapeutic treatment that requires active or facilitated transport across the target cells plasma membrane, and/or docks to, and/or chemically alters, and/or binds to biologic targets on the plasma membrane of the targeted cells. Examples include but are not limited to small molecules (that use facilitated uptake mechanisms), biologics—fusion-proteins, immunotoxins, antibodies, gene therapies and oncolytic virus or vaccines. The method is not useful for drugs that use passive diffusion to enter the target cells. This method enables treatment, or enhances efficacy of existing treatments through pre-dosing, dosing and post-dosing formulations, conditions and tissue conditioning. The pre-dosing and dosing formulations and conditions maximize drug-target binding interactions. The method includes distention of the target organ to mechanically stretch the tissue and thereby maximize exposure of targets on the cell membranes to targeted therapies. Post-dosing formulations and conditions facilitate post-binding events, such as intracellular uptake, release of drug from endosomes or lysosomes, or chaperone functions that enhance efficacy.

The present methods relate to the use of DT-EGF fusion proteins and other drugs that must interact with defined receptor structures exposed on surface of target cells—such as EGF receptors, tumor antigen or biomarkers—used in a manner that enables (i) maximum binding of the drug to its target, (ii) maximum binding specificity and uptake of drug by target cells, (iii), post-binding events including cellular uptake and release into the cytoplasm or other intracellular organelle, (iv) minimum time required to approach binding saturation (iv) optimal time and conditions to maximize efficacy (v), minimum non-specific off-target drug uptake and (vi) subsequent minimization of side effects and undesired toxicity. This provides a means of treating disease involving the tissues in distentable organs and specifically of utilizing DT-EGF drugs for treating disease associated with EGFR and/or related EGFR receptor family expression.

DT-EGF has been considered for development as a potential anticancer therapy for over twenty years¹⁻¹¹. In the few DT-EGF fusion proteins that have been constructed and tested, the naturally occurring DT sequence has had its binding domain replaced with the naturally occurring EGF sequence with 0-5 intervening amino acids acting as a linker. The impact of the linker of EGF domain upon the DT toxicity has not been assessed; however, it has been reported that the addition of the linker and DT sequence to the N-terminus of the EGF sequence has reduced its binding by up to 30-fold and its subsequent signal transduction.

The EGF-EGFR interactions have been well studied. In purified systems a number of mutants in the EGF sequence have demonstrated significant decreases in binding EGFR, while others have demonstrated increased binding and/or increased association rates¹². As noted above, the addition of the DT sequence has had a negative impact upon receptor binding by the EGF moiety.

While DT driven toxicity of DT-EGF constructs has been demonstrated in vitro and in vivo, the magnitude of the impact of the linker and EGF moiety upon the DT induced toxicity is unknown.

In some aspects, a linker sequence that is susceptible to lysosomal and or intracellular proteases that enable release of the EGF moiety will increase the toxicity of the DT moiety and thereby increase the efficacy of the DT-EGF drug construct.

Many people have received vaccination for Diphtheria and therefore possess varying levels of inactivating antibodies that can decrease the efficacy of DT-EGF constructs. By incorporating mutations in the sequence that are reported to decrease DT immunogenicity¹³, improvement in DT-EGF efficacy can be achieved.

Provided herein are combinations of the three elements mentioned above, (i) EGF mutants to improve binding, (ii) linker sequence to minimize DT, EGF cross-inhibition (e.g. as in the case where linking caused a 20-fold reduction in the affinity of the EGF moiety to EGFR) or enable intracellular release of DT (e.g a cleavable linker that releases the DT moiety once inside the target cell), and (iii) mutations that de-immunize the DT moiety.

Minor changes, including single amino acid residue substitutions, can have profound effects on activity in unpredictable ways; thus, the method outlined above to improve DT-EGF construct activity and efficacy will enable reasonable predictions but will ultimately require empirical validation. Several mutations were in the EGF moiety were attempted, and most failed to exhibit improved EGFR binding.

The following sequence is considered the standard that contains essentially wild type DT and EGF sequences joined by a sequence of amino acids that is amenable to in vivo activity but with a known 30-fold reduction in EGF to EGFR binding affinity: the amino acid sequence of protein 1 (A-dmDT390-EGF), a fusion of a truncated Diphtheria toxin with Epidermal Growth Factor.

Information on the sequences include: 1, Additional alanine residue; 2˜391,dmDT390; 19, S to A (mutation for removal of N-glycosylation site); 236, N to A (mutation for removal of N-glycosylation site); 392-393, amino acid sequence for NcoI site; 394-446: EGF
agaddvvdss ksfvmenfas yhgtkpgyvd siqkgiqkpk sgtqgnyddd wkgfystdnk ydaagysvdn enplsgkagg vvkvtypglt kvlalkvdna etikkelgls lteplmeqvg teefikrfgd gasrvvlslp faegsssvey innweqakal sveleinfet rgkrgqdamy eymaqacagn rvrrsvgssl scinldwdvi rdktktkies lkehgpiknk msespa ktvs eekakqylee fhqtalehpe lselktvtgt npvfaganya awavnvaqvi dsetadnlek ttaalsilpg igsvmgiadg avhhnteeiv aqsialsslm vaqaiplvge lvdigfaayn fvesiinlfq vvhnsynrpa yspghktqpf lpwnsdsecp lshdgyclhd gvcmyieald kyacncvvgy igercqyrdl kwwelr (SEQ ID NO: 5)

Too often drugs benefit only a small fraction of patients with relapse common in responders. The current invention aims to increase the fraction of responders and decrease the frequency of relapse through a method that enhances existing drug efficacy or enabling optimal efficacy in novel candidate drugs.

Drugs have specificity for disease cells or are designed to specifically target elements of the diseased cells. All drugs, regardless of mechanism, must bind and/or be taken up by their respective target cell of interest. Unfortunately, in the practical settings of disease treatment, drugs rarely if ever bind all targets or achieve uniform targeting of the whole population of diseased cells. This is one cause of incomplete response and recurrence of disease. In one aspect, use of pre-dosing and dosing conditions including benign distention of the target tissue to achieve maximum target binding enhances therapeutic benefit. In another aspect, post-dosing instillation of a formulation under conditions designed to enhance post-drug binding events improves drug activity and efficacy.

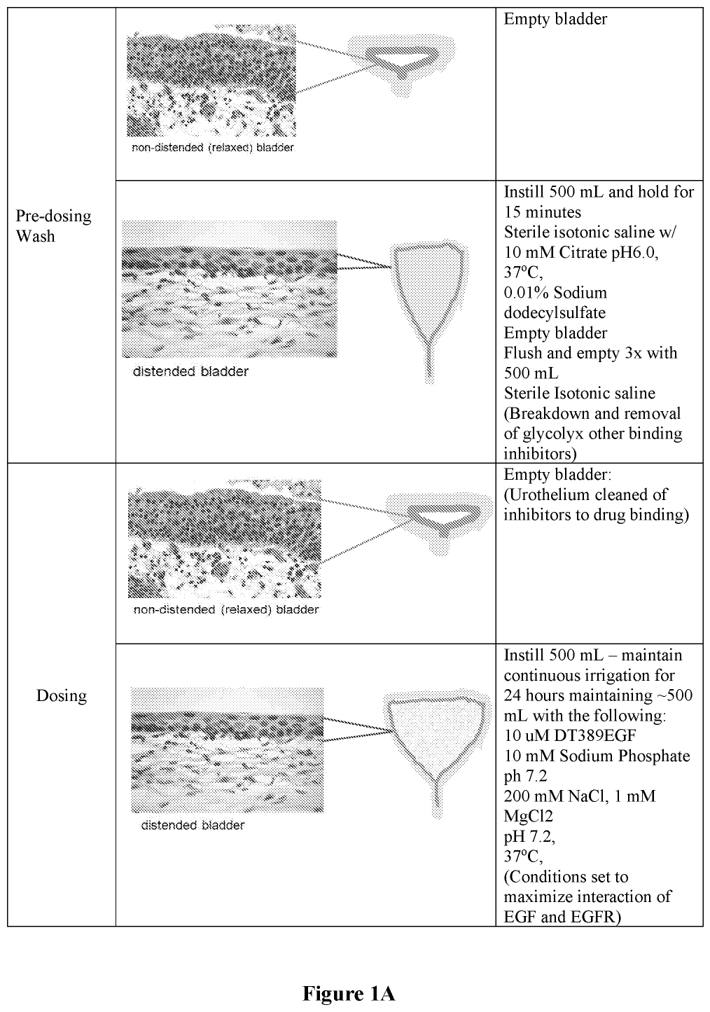

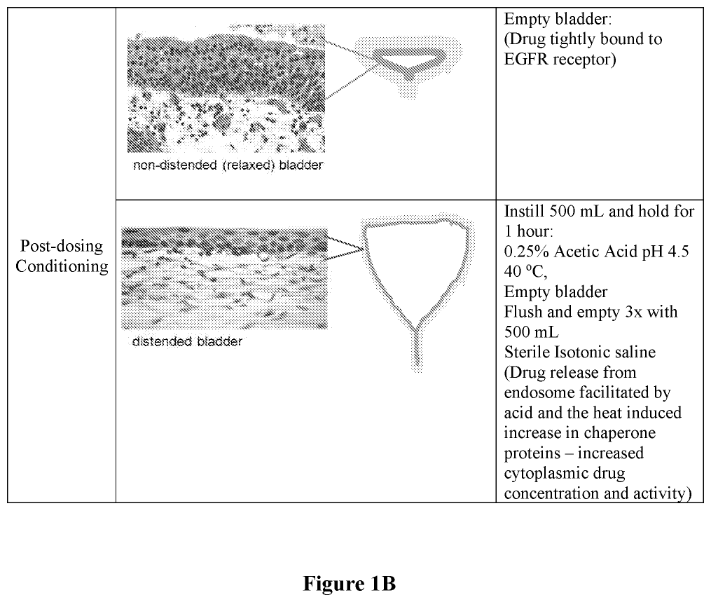

The potential to hold volume for a set period of time within an organ or tissue—a ‘distentable tissue’—is currently used to treat disease. However, intentional distending of an organ or tissue, such as a bladder, to increase its drug uptake has not been reported or proposed. Intravesicular treatments for bladder cancer are presented as examples of the current approaches being used and to contrast the differences and benefits enabled by the current invention; the concept can be extended to any distentable tissue. A healthy adult will typically empty their bladder when it has filled to between 250-500 mL; however, the bladder may hold up to 1000 mL without damage. The shape of the bladder and the interior surface area obviously change greatly during filling and transition from empty to distended (250 mL-1000 mL). The urothelium lining the interior of the bladder is transitional epithelium, so named, because it can transition from a compacted structure to an extended structure while maintaining its integrity. The layers of transitional epithelial cells of the urothelium undergo extreme shape changes as the bladder fills and ultimately the superficial lining cells appear irregular and squamous as they are stretched and expose their greatest surface area—see FIG. 1. It is this mechanism that the present disclosure exploits to enable the requisite interactions of drug with cell surface structures, which surprisingly and unexpectedly maximizes drug activity.

With current therapies for bladder cancer and those in clinical testing, volumes of 20-75 mL are intravesicularly delivered into an empty bladder and then held within the bladder for 2 hours; this is true for small molecules therapeutics (e.g. Gemcytabine, apaziquone), biologics proteins (e.g. VB4-845) or nucleic acid based gene therapies (e.g. BC819) or oncolytic virus (e.g. CG0700, rAd-IFN).

A pre-dosing wash of the bladder contents can improve the ability of a drug to bind targets on the bladder lining—urothelium. One existing approach demonstrating improved uptake of a potential therapeutic oncolytic virus is the disruption or removal of the glycocalyx. The glycocalyx is a glycoprotein layer between the uroepithelial cell layer and the contents of the bladder. Viral infection of the urothelium can be increased by flushing the bladder with a mild detergent solution designed to remove or disrupt the glycocalyx¹⁴. An example of this is the prewash used prior to instilled treatment with rAD-IFN virus—currently in clinical trials to treat bladder cancer. While the pre-wash condition used prior to CG0700-rAD-INF treatment may benefit other drugs with different uptake mechanisms, different prewash formulations and conditions improve binding and uptake of those different drugs. It has been demonstrated in a different system that disruption of the glycocalyx by treatment with neuraminidase facilitates HLA/anti-HLA-mediated cell attachment: a 3.6-fold increase of the two-dimensional binding constant was reported¹⁵ The goal of a prewash is to remove factors like the glycocalyx that would interfere with the binding and uptake of drug while simultaneously minimizing any damage or change to the urothelium that could be harmful; the balance between these two requires empirical determination, and the prewash provided herein is surprisingly effective.

A very small range (20-75 mL) of drug solution volumes are typically instilled in the bladder; no clinical investigation of efficacy as a function of volume has been presented. A study in mice reported no difference in virus uptake in the bladder urothelium between a 50 and 100 uL instilled volume; (it was not noted that both volumes are well below the >150 uL needed for pronounced bladder distention in mice). Distention has not previously been proposed to increased accessibility of binding sites for drugs or other binding interactions. The present disclosure provides that maximizing the surface area of the target tissue by distention enables access to the maximum number of target sites for drug entry into the cell. Moreover, in the case of bladder distention an added and surprising benefit is induction of reduced flow from the ureters and subsequent minimization of the changes to the binding buffer/solution instilled into the bladder.

The constituents of urine: water, urea, creatinine, proteins, hormones, inorganic salts and other organic compounds with a range in pH of about 5.5-7, negatively impact binding interactions between disease cells and the treatments designed to kill or modify them.

In some aspects, utilization of a dosing solution for instillation is provided, such that binding conditions are maximized and maintained for a sufficient time to approach saturation of binding sites.

Elements of instillation to be utilized include the following: time, volume, temperature, pH, viscosity, dielectric, ionic strength, monovalent ions, divalent ions, excipients that act as volume excluders, detergents, chaotropic agents, stabilizers. Colorization agents used to monitor and assess the degree of dilution caused by addition or urine during the treatment time.

The optimal dosing solution with best conditions and time required for ideal binding cannot be predetermined. The need to empirically determine—with purified systems, cell-based testing or in vivo models—optimal formulations and conditions is a function of the complexity of system. The optimal conditions reported for EGF binding to EGFR have demonstrated that even small changes in ligand proteins or their receptor sequences significantly and measurably change binding in ways that cannot be predicted (Cochran et al. U.S. Pat. No. 8,865,864 B2). Relatively small changes in a sequence have significant and measurable differences in binding on- and off-rates and affinity. The N-terminal addition of long sequences, such as the diphtheria toxin used in the DT389EGF fusion-toxin¹⁶ can affect the binding to the EGFR. Essentially all biological interactions of interest (e.g., ligand-receptor, protein-protein interaction, small molecule-transport proteins and transmembrane transport of various compounds) have complex dependency on pH, temperature, concentration, viscosity, dielectric constant, ionic strength and solute concentration of most any type. This indicates the need to empirically determine the optimal binding conditions for any given change in sequence or environment.

Large changes such as attachment of antibody or toxin sequence elicits unknown changes in the requirements for the following conditions to demonstrate affinity of ligand for receptor: temperature, ligand concentration, monovalent and divalent concentration, choatropic excipient concentration, need for prewash, pH, viscosity and binding time.

A report of the bladder-cancer cell killing ability of DT398EGF showed that 24-hour exposure of drug caused more cell death than a 2-hour pulse of the drug¹⁷. However, the minimal or optimal time required was not investigated in cell culture and thus has not been a parameter tested in the in-vivo setting. As noted above, convention for current oncology therapies is a 2-hour hold of an instilled drug formulation. While the time required for binding in a given formulation and under specified conditions in a clinical setting cannot be predicted, we can set boundaries to initiate empirical optimization. Reports measuring binding rates in purified EGF/EGFR systems indicate complete binding on the minutes time scale, while the cell based system above indicates hours to days may be needed. As such, a time point between 10 minutes and 48 hours is contemplated herein to drive optimal efficacy. If more than 2-hours are needed, continuous irrigation is used. The flow rates into and out of the bladder, and the bladder volume can be maintained such that constant drug concentrations (within practical ranges and above the determined therapeutic threshold) and formulation conditions can remain optimal for the required amount of time using current clinical procedures used by those skilled in the art.

The above discussion emphasized the parameters which impact and allow improved efficacy of the binding events themselves. Pre-dosing, dosing and post-dosing conditions upon the target organ and target cells also impact the efficacy or reduction in toxicity of many treatments. Reports¹⁸ have demonstrated that the acidification of lysosomes is important to diphtheria toxin release into the cytoplasm; acidification of the cell media increases toxicity while blocking acidification reduces or prevents toxicity. Thus, pH is important to more than just binding. Reports¹⁹ have also demonstrated the heat shock proteins are important chaperone proteins that aid in the translocation of diphtheria toxin from endosomes into the cytoplasm. Thus, temperature—and its use to induce increased chaperone protein activity—may be used to increased toxicity through mechanisms unrelated to binding. Thus, exposure to altered temperatures and pH by the target organ and/or cells for time prior, during or post-dosing can have significant effects upon activity by protein-toxin fusion therapies and other therapies.

Provided herein are compositions and methods for treating disease that has overexpression of EFG-receptors on its cell surface by administering DT-EGF fusion proteins to a subject in need thereof. In one aspect, the disclosed methods may reduce the immune response caused by the sequences of DT by altering the amino acid is such a way that does not significantly affect DT-induced toxicity. In another aspect, the disclosed methods may increase the affinity of the EGF moiety of DT-EGF constructs for the EGF receptors. In another aspect, the disclosed methods may insert amino acids sequence between the DT and EGF moieties to minimize any antagonism—such as reduce EGFR binding or reduced DT-induced toxicity—or enable release of DT from the DT-EGF construct once inside the target cell.

In one embodiment the method combines the naturally occurring DT-sequence with the binding domain replaced with a short sequence of 0-20 amino acids to EGF that has been modified with any of the mutations reported to increase EGF-receptor association rate or affinity. An exemplary embodiment includes the amino acid sequence of protein (A-dmDT390-EGF-Kd+), a fusion of a truncated Diphtheria toxin with an enhanced binding Epidermal Growth Factor mutant.

Mutations to the AXYZ sequence below are indicated with capital letters.
agaddvvdss ksfvmenfas yhgtkpgyvd siqkgiqkpk sgtqgnyddd wkgfystdnk ydaagysvdn enplsgkagg vvkvtypglt kvlalkvdna etikkelgls lteplmeqvg teefikrfgd gasrvvlslp faegsssvey innweqakal sveleinfet rgkrgqdamy eymaqacagn rvrrsvgssl scinldwdvi rdktktkies lkehgpiknk msespa ktvs eekakqylee fhqtalehpe lselktvtgt npvfaganya awavnvaqvi dsetadnlek ttaalsilpg igsvmgiadg avhhnteeiv aqsialsslm vaqaiplvge lvdigfaayn fvesiinlfq vvhnsynrpa yspghktqpf lpwnsYsecp PsYdgyclhd gvcRyieald Syacncvvgy Agercqyrdl RwwGRr (SEQ ID NO: 6)

In some aspects, Z is an EGF with modifications improving EGFR binding characteristics. For example, Z can be:
wnsYsecp PsYdgyclhd gvcRyieald Syacncvvgy Agercqyrdl RwwGRr (SEQ ID NO: 3)

In one embodiment the method combines incorporation or reported sequence modifications that reduce immune recognition of DT-sequence with the binding domain replaced with a short sequence of 0-20 amino acids to EGF that retains the naturally occurring amino acid sequence.

In one embodiment the method combines incorporation or reported sequence modifications that reduce immune recognition of DT-sequence with the binding domain replaced with a short sequence of 0-20 amino acids to EGF that has been modified with any of the mutations reported to increase EGF-receptor association rate or affinity.

In one embodiment the method combines the naturally occurring DT-sequence with the binding domain replaced with a short sequence of 0-20 amino acids designed to be targeted by lysosomal or intracellular proteases—to EGF that has been modified with any of the mutations reported to increase EGF-receptor association rate or affinity.

In one embodiment, the method combines incorporation or reported sequence modifications that reduce immune recognition of DT-sequence with the binding domain replaced with—a short sequence of 0-20 amino acids designed to be targeted by lysosomal or intracellular proteases—to EGF that retains the naturally occurring amino acid sequence.

In one embodiment the method combines incorporation or reported sequence modifications that reduce immune recognition of DT-sequence with the binding domain replaced with a short sequence of 0-20 amino acids designed to be targeted by lysosomal or intracellular proteases—to EGF that has been modified with any of the mutations reported to increase EGF-receptor association rate or affinity.

In one aspect, provided herein is a method to treat disease in a distentable organ in a subject, the method comprising administering into a distentable organ or tissue of the subject with a pharmaceutical composition comprising a formulation disclosed herein and using a dosing regimen designed to improve drug-target interaction and overall efficacy. The disclosed methods may pretreat a distentable organ with a formulation and set of conditions that increases and improves the binding and affinity of the drug to its target and/or improves efficacy by secondary mechanisms. The disclosed methods may treat a distentable organ with a drug formulation and set of conditions that increases and improves the binding and affinity of the drug to its target and/or improves efficacy by secondary mechanisms. The disclosed methods may post-treat a distentable organ with a formulation and set of conditions that increases and improves the binding and affinity of the drug to its target and/or improves efficacy by secondary mechanisms. The disclosed methods use pre-dosing, dosing and post-dosing formulations and conditions may include volume, temperature, pH, viscosity, dielectric, ionic strength, monovalent ions, divalent ions, excipients that act as volume excluders, detergents, chaotropic agents, stabilizers.

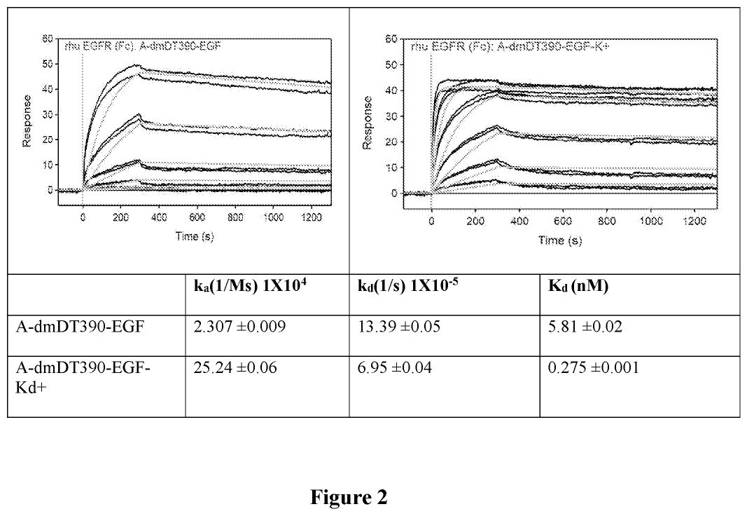

In one aspect, provided herein is a method to treat disease in a distentable organ in a subject. The method comprises administering into the organ of the subject a pharmaceutical composition comprising formulations as demonstrated to improve drug-target interaction and overall efficacy (FIGS. 2-5). The methods may use any of the set of conditions shown to increase and improve the binding and affinity of the drug to its target and/or improve efficacy. FIGS. 2-5 experimentally demonstrate the following conditions enable binding between A-dmDT390-EGF (of SEQ ID NO: 1) or A-dmDT390-EGF-Kd+(of SEQ ID NO: 2) and EGFR:
HBS (10 mM HEPES pH 7.4, 150 mM NaCl, and 0.05% Surfactant P20)
PBS (10 mM Sodium Phosphate pH 7.4, 150 mM NaCl, 0.05% Surfactant P20)
150-500 mM NaCl
0-20 mM MgCl₂
10-500 mM NaPO₄
0-200 mM Glucose
RMPI (all constituents)
0-1 mM EDTA
pH 6-8.

One of skill in the art would not have expected to see pH 7.4 was so significantly improved as compared to pH 6 (especially given that urine typically exhibits a pH below 6. Also surprising was the improved binding in the absence of divalents such as MgCl₂, or with minimal amounts of divalents, for example, less than about 20 mM, or less than about 10 mM, or less than about 2 mM divalents. And finally, the improved binding with increasing glucose would not be expected by one of skill in the art.

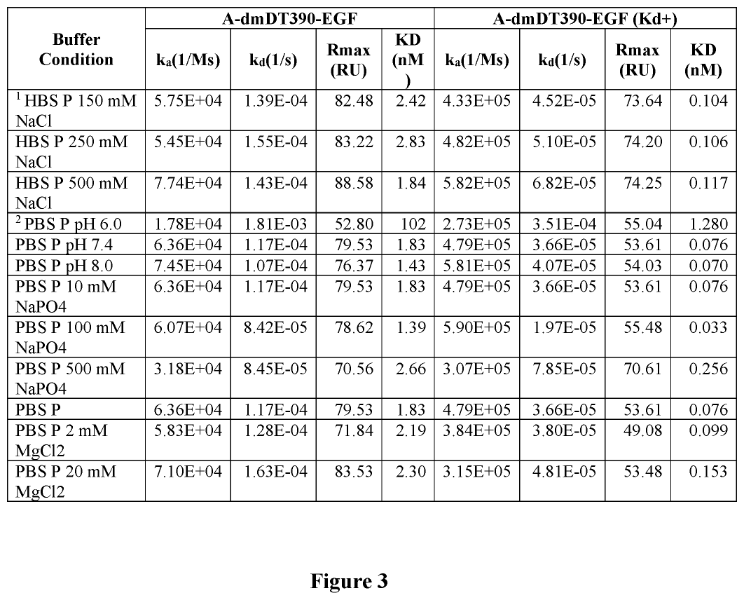

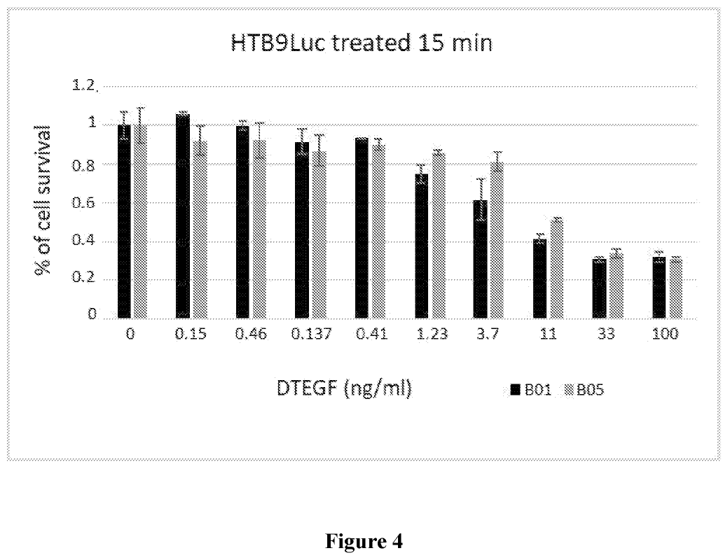

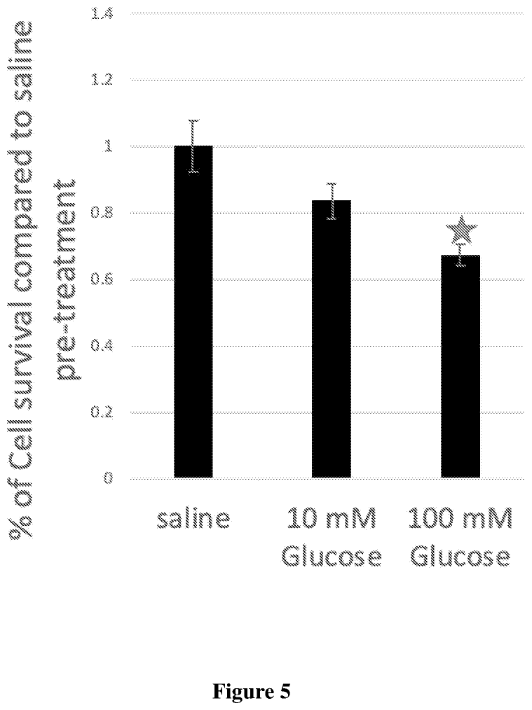

First demonstrated herein, a pH at or above 7.4 provides improved binding (FIG. 3), for example at least about pH 7.0, about 7.2, about 7.4, about 7.6, about 7.8, about 8.0, and so on, and from FIG. 4, monovalent ions, such as NaCl show an optimum of the tested conditions found at about 10 mM, for example, about 5 mM to about 15 mM, or about 8 mM to about 12 mM, or about 5 mM, or about 6 mM, or about 7 mM, or about 8 mM, or about 9 mM, or about 10 mM, or about 11 mM, or about 12 mM, or about 13 mM, or about 14 mM, or about 15 mM, while the ionic strength of buffering agents, such as NaPO₄ are effective with an optimum of the tested conditions found at about 150 mM, for example, at about 145 mM, about 148 mM, about 152 mM, about 154 mM, at about 140 to about 160 mM, or about 145 mM to about 155 mM, or about 150 mM to about 160 mM, or about 140 mM to about 150 mM, and so on, and divalent ions, such as MgCl₂ are most effective if they are absent. The absence of divalent ions can be achieved by using chelation agents such as bisphosphonates or EDTA (as was present up to 35 uM in the drug solution of SPR experiments). The presence of about 10 mM to about 150 mM glucose, or about 20 mM to about 120 mM glucose, or at least about 10 mM glucose, at least about 50 mM glucose, or at least about 100 mM glucose improved binding, for example, in the tested conditions shown in FIG. 5.

In one embodiment, the method comprises treating the subject with bladder cancer with an instilled formulation comprising: 10 mM PO₄, 150 mM NaCl, pH 8.0.

In one embodiment, the method comprises treating the subject with bladder cancer with an instilled formulation comprising: 10 mM PO₄, 150 mM NaCl, pH 8.0, 50-200 mM Glucose.

In one embodiment, the method comprises treating the subject with bladder cancer with an instilled formulation comprising: 10 mM PO₄, 150 mM NaCl, pH 8.0, 0.5-20 mM EDTA.

In one embodiment, the method comprises treating the subject with bladder cancer with an instilled formulation comprising: 10 mM PO₄, 150 mM NaCl, pH 8.0, 20 mM zolendronate (or any bisphosphonate).

In one aspect, provided herein is a method providing colorization agents used to monitor and assess the degree of dilution caused by addition or urine during the treatment time and to maintain conditions within optimal ranges.

In one aspect, provided herein is a method that uses known or published binding conditions as elements of strategies for improving efficacy in formulations and conditions development, or as elements of the formulations and/or conditions themselves.

In one aspect, provided herein is a method that uses cell-based assays and/or in vivo assessment of binding conditions as elements of strategies for improving efficacy in formulations and conditions development, or as elements of the formulations and/or conditions themselves.

In one aspect, provided herein is a method that uses cell-bases assays and/or in vivo assessment of pre-dosing, dosing, and post-dosing conditions as elements of strategies for improving efficacy in formulations and conditions development, or as elements of the formulations and/or conditions themselves.

In one embodiment the method comprises treating the subject with bladder cancer with an instilled volume (75 mL-1500 mL) that maximizes exposure of cellular surface targets to a fusion protein-toxin, such as Diphtheria-EGF to its EGFR target, or epCAM-ToxinA to its EpCAM target.

In one embodiment, the method comprises treating the subject with bladder cancer with an instilled volume (75 mL-1500 mL) that minimizes dilution of the drug formulation designed to improve binding to cellular surface targets by a fusion protein-toxin drug.

In one embodiment, the method comprises treating the subject with bladder cancer with an instilled formulation and conditions that maximizes binding to cellular surface targets by a fusion protein-toxin drug, such as Diphtheria-EGF to its EGFR target, or epCAM-ToxinA to its EpCAM target.

In one embodiment, the method comprises treating the subject with bladder cancer with a pre-dose instilled formulation under conditions that maximizes binding to cellular surface targets by a fusion protein-toxin drug, such as Diphtheria-EGF to its EGFR target, or epCAM-ToxinA to its EpCAM target, by disruption or removal of the glycocalyx or other inhibitors of binding.

In one embodiment, the method comprises treating the subject with bladder cancer with a pre-dose instilled formulation under conditions that maximizes binding to cellular surface targets by a fusion protein-toxin drug, such as Diphtheria-EGF to its EGFR target, or epCAM-ToxinA to its EpCAM target, and/or facilitates secondary mechanisms (other than binding enhancement) that improve efficacy.

In one embodiment, the method comprises treating the subject with bladder cancer with a post-dose instilled formulation under conditions that maximizes binding to cellular surface targets by a fusion protein-toxin drug, such as Diphtheria-EGF to its EGFR target, or epCAM-ToxinA to its EpCAM target, and/or facilitates secondary mechanisms (not binding enhancement) that improve efficacy.

In one embodiment, the method comprises treating the subject with bladder cancer with a pre-dosing and/or dosing and/or post-dose instilled formulation with a instillate hold-time to maximize binding to cellular surface targets by a fusion protein-toxin drug, such as Diphtheria-EGF to its EGFR target, or epCAM-ToxinA to its EpCAM target, and/or facilitates secondary mechanisms (not binding enhancement) that improve efficacy.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1

Binding measurements and analyses of two DTEGF fusion-toxins to EGF by Surface Plasmon Resonance (SPR) were determined. The data was collected using a 300 (s) association phase and a 900 (s) dissociation phase. Kinetic rate coefficients were recovered from a binding analysis experiment performed with a Biacore 3000 biosensor. Six concentrations of analyte ranging between 300 nM to 1.23 nM were run in duplicate against captured rhu EGFR (Fc). The association and dissociation phase data were globally fit to a 1:1 binding model to determine the association rate coefficient (ka), dissociation rate coefficient (kd) and the Rmax value. Results are reported as the global fits to 1:1 binding model±standard deviation. See FIG. 2.

### Example 2

SPR assay to screen buffer conditions—NaCl, pH, buffer strength, and divalent cation for the A-dmDT390-EGF and A-dmDT390-EGF-Kd+/rhuEGFR interactions. The rhuEGFR capture at ˜275 RU. A single of 300 nM concentration of each analyte was injected. ¹HBS P 10 mM Hepes, 150 mM NaCl, 0.05% Surfactant P20, pH 7.4; ²PBS P 10 mM Sodium Phosphate, 150 mM NaCl, 0.05% Surfactant P20, pH 7.4. See FIG. 3.

### Example 3

A-dmDT390-EGF (B05) and A-dmDT390-EGF-Kd+(BO1) treatments for 15 minutes demonstrate potent cell killing on bladder cancer cell line HTB9. See FIG. 4.

### Example 4

HTB9 cells seeded in 96 wells plate on day 1 in RPMI1640 with 10% FCS, attached for 2 days then treated with 5 ng/mL A-dmDT390-EGF with the indicated glucose concentration for 2 hours. Removed the treatment, washed twice with 50 ul medium then added 100 ul medium and incubate for 72 hours, viability was accessed with MTT assay. Each condition was done with triplicate. See FIG. 5.

### REFERENCES

1. Cohen K A, Liu T F, Cline J M, et al: Toxicology and pharmacokinetics of DT388IL3, a fusion toxin consisting of a truncated diphtheria toxin (DT388) linked to human interleukin 3 (IL3), in cynomolgus monkeys. Leuk Lymphoma 45:1647-56, 2004
2. Cohen K A, Liu T F, Cline J M, et al: Safety evaluation of DT388IL3, a diphtheria toxin/interleukin 3 fusion protein, in the cynomolgus monkey. Cancer Immunol Immunother 54:799-806, 2005
3. Horita H, Thorburn J, Frankel A E, et al: EGFR-targeted diphtheria toxin stimulates TRAIL killing of glioblastoma cells by depleting anti-apoptotic proteins. J Neurooncol 95:175-184, 2009
4. Liu T F, Cohen K A, Ramage J G, et al: A diphtheria toxin-epidermal growth factor fusion protein is cytotoxic to human glioblastoma multiforme cells. Cancer Res 63:1834-7, 2003
5. Liu T F, Cohen K A, Willingham M C, et al: Combination fusion protein therapy of refractory brain tumors: demonstration of efficacy in cell culture. J Neurooncol 65:77-85, 2003
6. Liu T F, Hall P D, Cohen K A, et al: Interstitial diphtheria toxin-epidermal growth factor fusion protein therapy produces regressions of subcutaneous human glioblastoma multiforme tumors in athymic nude mice. Clin Cancer Res 11:329-34, 2005
7. Liu T F, Tatter S B, Willingham M C, et al: Growth factor receptor expression varies among high-grade gliomas and normal brain: epidermal growth factor receptor has excellent properties for interstitial fusion protein therapy. Mol Cancer Ther 2:783-7, 2003
8. Liu T F, Urieto J O, Moore J E, et al: Diphtheria toxin fused to variant interleukin-3 provides enhanced binding to the interleukin-3 receptor and more potent leukemia cell cytotoxicity. Exp Hematol 32:277-81, 2004
9. Liu T F, Willingham M C, Tatter S B, et al: Diphtheria toxin-epidermal growth factor fusion protein and Pseudomonas exotoxin-interleukin 13 fusion protein exert synergistic toxicity against human glioblastoma multiforme cells. Bioconjug Chem 14:1107-14, 2003
10. Mishra G, Liu T F, Frankel A E: Recombinant toxin DAB389EGF is cytotoxic to human pancreatic cancer cells. Expert Opin Biol Ther 3:1173-80, 2003
11. Yang X, Kessler E, Su L J, et al: Diphtheria toxin-epidermal growth factor fusion protein DAB389EGF for the treatment of bladder cancer. Clin Cancer Res 19:148-57, 2013
12. Lahti J L, Lui B H, Beck S E, et al: Engineered epidermal growth factor mutants with faster binding on-rates correlate with enhanced receptor activation. FEBS Lett 585:1135-9, 2011
13. Schmohl: Mutagenic Deimmunization of Diphtheria Toxin for use in Biological Drug Development. Toxins 7:4067-4082, 2015
14. Ramesh N, Memarzadeh B, Ge Y, et al: Identification of pretreatment agents to enhance adenovirus infection of bladder epithelium. Mol Ther 10:697-705, 2004
15. Saitakis M, Gizeli E: Quantification of the effect of glycocalyx condition on membrane receptor interactions using an acoustic wave sensor. Eur Biophys J 40:209-15, 2011
16. Liu T F, Cohen K A, Ramage J G, et al: A diphtheria toxin-epidermal growth factor fusion protein is cytotoxic to human glioblastoma multiforme cells. Cancer Res 63:1834-7, 2003
17. Yang X, Kessler E, Su L J, et al: Diphtheria toxin-epidermal growth factor fusion protein DAB389EGF for the treatment of bladder cancer. Clin Cancer Res 19:148-57, 2013
18. Lemichez E, Bomsel M, Devilliers G, et al: Membrane translocation of diphtheria toxin fragment A exploits early to late endosome trafficking machinery. Mol Microbiol 23:445-57, 1997
19. Schuster M, Schnell L, Feigl P, et al: The Hsp90 machinery facilitates the transport of diphtheria toxin into human cells. Sci Rep 7:613, 2017

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicant to relate to each and every individual publication, database entry (e.g., Genbank sequences or GeneID entries), patent application, or patent identified even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1. A composition comprising:
a. A diphtheria toxin-epidermal growth factor (DT-EGF) fusion protein of structure A-X-Y-Z, wherein:
b. A is 0-5 amino acid residue N-terminal addition to the front of the diphtheria toxin sequence;
c. X is a diphtheria toxin fragment or mutated fragment that maintains catalytic activity;
d. Y is sequence of amino acids from 0-20 aa in length that connects the carboxy terminus of X to the amino terminus of Z; and
e. Z is epidermal growth factor or mutant thereof that maintains binding affinity for epidermal growth factor receptor.

2. The composition of claim 1, wherein the composition is in a formulation having a pH of at least about 7.0, at least about 7.4, or at least about 8.

3. The composition of claim 1, wherein the composition is in a formulation that comprises at least about 10 mM glucose, at least about 50 mM glucose, or at least about 100 mM glucose.

4. The composition of claim 1, wherein the composition in in a formulation having a minimal amount of MgCl₂, or less than about 20 mM MgCl₂, or less than about 10 mM MgCl₂, or less than about 2 mM MgCl₂.

5. The composition of claim 1, wherein the composition is formulated with 10 mM PO₄ and 150 mM NaCl at pH 8.0.

6. The composition of claim 1, where A is a single alanine.

7. The composition of claim 1, where X is (glycosylation sites removed)
gaddvvdss ksfvmenfas yhgtkpgyvd siqkgiqkpk sgtqgnyddd
wkgfystdnk ydaagysvdn enplsgkagg vvkvtypglt kvlalkvdna etikkelgls lteplmeqvg teefikrfgd gasrvvlslp faegsssvey innweqakal sveleinfet
rgkrgqdamy eymaqacagn rvrrsvgssl scinldwdvi rdktktkies lkehgpiknk msespaktvs eekakqylee fhqtalehpe lselktvtgt npvfaganya awavnvaqvi dsetadnlek ttaalsilpg igsvmgiadg avhhnteeiv aqsialsslm vaqaiplvge
lvdigfaayn fvesiinlfq vvhnsynrpa yspghktqpf (SEQ ID NO: 1).

8. The composition of claim 1 where X is
gaddvvdss ksfvmenfss yhgtkpgyvd siqkgiqkpk sgtqgnyddd
wkgfystdnk ydaagysvdn enplsgkagg vvkvtypglt kvlalkvdna etikkelgls lteplmeqvg teefikrfgd gasrvvlslp faegsssvey innweqakal sveleinfet
rgkrgqdamy eymaqacagn rvrrsvgssl scinldwdvi rdktktkies lkehgpiknk msespnktvs eekakqylee fhqtalehpe lselktvtgt npvfaganya awavnvaqvi dsetadnlek ttaalsilpg igsvmgiadg avhhnteeiv aqsialsslm vaqaiplvge lvdigfaayn fvesiinlfq vvhnsynrpa yspghktqpf (SEQ ID NO: 2)

9. The composition of claim 1 where X is a modified sequence that maintains catalytic activity while also evading immune response.

10. The composition of claim 1 where Y is
a. ha,
b. pw,
c. aa,
d. lp, or
e. gg.

11. The composition of claim 1 where Y is
a. A short sequence of 0-20 amino acids designed to be targeted by lysosomal or intracellular proteases
b. A short sequence of 0-20 amino acids designed to minimize negative or inhibitory interactions of X and Z sequences.

12. The composition of claim 1 where Z is wild type human EGF sequence.

13. The composition of claim 1 where Z is:
a. wnsYsecp PsYdgyclhd gvcRyieald Syacncvvgy Agercqyrdl RwwGRr (SEQ ID NO: 3); or
b. any mutant that increases affinity for the EGF receptor.

14. The composition of claim 1 comprising:
agaddvvdss ksfvmenfas yhgtkpgyvd siqkgiqkpk sgtqgnyddd wkgfystdnk
ydaagysvdn enplsgkagg vvkvtypglt kvlalkvdna etikkelgls lteplmeqvg teefikrfgd
gasrvvlslp faegsssvey innweqakal sveleinfet rgkrgqdamy eymaqacagn
rvrrsvgssl scinldwdvi rdktktkies lkehgpiknk msespaktvs eekakqylee fhqtalehpe
lselktvtgt npvfaganya awavnvaqvi dsetadnlek ttaalsilpg igsvmgiadg avhhnteeiv
aqsialsslm vaqaiplvge lvdigfaayn fvesiinlfq vvhnsynrpa yspghktqpf lpwnsYsecp PsYdgyclhd gvcRyieald Syacncvvgy Agercqyrdl RwwGRr (SEQ ID NO: 6).

15. The composition of claim 1 comprising:
agaddvvdss ksfvmenfas yhgtkpgyvd siqkgiqkpk sgtqgnyddd wkgfystdnk
ydaagysvdn enplsgkagg vvkvtypglt kvlalkvdna etikkelgls lteplmeqvg teefikrfgd gasrvvlslp faegsssvey innweqakal sveleinfet rgkrgqdamy eymaqacagn
rvrrsvgsslscinldwdvi rdktktkies lkehgpiknk msespaktvs eekakqylee fhqtalehpe
lselktvtgt npvfaganya awavnvaqvi dsetadnlek ttaalsilpg igsvmgiadg avhhnteeiv aqsialsslm vaqaiplvge lvdigfaayn fvesiinlfq vvhnsynrpa yspghktqpf lpwnsdsecp lshdgyclhd gvcmyieald kyacncvvgy igercqyrdl kwwelr (A-dmDT390-EGF) (SEQ ID NO: 5).

16. A method to treat cancer in a distentable organ with an instilled volume:
(i) comprising maximizing exposure of cellular surface targets to a targeted drug treatment;
(ii) comprising maximizing exposure of cellular surface targets to a targeted drug treatment wherein the instilled volume comprises a formulation;
(iii) comprising a pre-dosing treatment that maximizes exposure of cellular surface targets to a targeted drug treatment wherein the instilled volume comprise a formulation;
(iv) comprising a pre-dosing treatment that removes constituents that inhibit binding and exposure of cellular surface targets to a targeted drug treatment wherein the instilled volume comprise a formulation;
(v) comprising a pre-dosing treatment that maximizes treatment efficacy of a targeted drug treatment by mechanisms other than improved binding of drug to target wherein the instilled volume comprise a formulation; or
(vi) comprising a post-dosing treatment that maximizes treatment efficacy of a targeted drug treatment by mechanisms other than improved binding of drug to target wherein the instilled volume comprise a formulation.

17. (canceled)

18. (canceled)

19. (canceled)

20. (canceled)

21. (canceled)

22. The method of claim 16, wherein the cancer is in the bladder, in the pleura, in the uterus, in the peritoneum, in the omentum, or in the eye.

23. (canceled)

24. (canceled)

25. (canceled)

26. (canceled)

27. (canceled)

28. The method of claim 16, where the targeted drug treatment is a protein-toxin fusion of Diphtheria toxin to Epidermal Growth factor.

29. The method of claim 16, where the targeted drug treatment is a protein-toxin fusion of Pseudomonas ExoToxin A to EpCAM.

30. The method of claim 16, where the targeted drug treatment is a protein-toxin fusion is ONTAK.

31. (canceled)

32. (canceled)

33. (canceled)


SEQUENCE LISTING (6 sequences)

SEQ ID NO: 1 - protein, 389 aa, Artificial Sequence
  Synthetic
GADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPF

SEQ ID NO: 2 - protein, 389 aa, Artificial Sequence
  Synthetic
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPF

SEQ ID NO: 3 - protein, 54 aa, Artificial Sequence
  Synthetic
WNSYSECPPSYDGYCLHDGVCRYIEALDSYACNCVVGYAGERCQYRDLRWWGRR

SEQ ID NO: 4 - protein, 445 aa, Artificial Sequence
  Synthetic
GADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWNSYSECPPSYDGYCLHDGVCRYIEALDSYACNCVVGYAGERCQYRDLRWWGRR

SEQ ID NO: 5 - protein, 446 aa, Artificial Sequence
  Synthetic
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWNSDSECPLSHDGYCLHDGVCMYIEALDKYACNCVVGYIGERCQYRDLKWWELR

SEQ ID NO: 6 - protein, 446 aa, Artificial Sequence
  Synthetic
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWNSYSECPPSYDGYCLHDGVCRYIEALDSYACNCVVGYAGERCQYRDLRWWGRR